(19) United States
(12) Patent Application Publication
(10) Pub. No.: US 2010/0285039 A1
(43) Pub. Date: Nov. 11, 2010

(54) B7-H1 (CD274) Antagonists Induce Apoptosis of Tumor Cells

(75) Inventor: Lieping Chen, Sparks Glencoe, MD (US)

Correspondence Address: JOHNS HOPKINS TECHNOLOGY TRANSFER, 100 N. CHARLES ST., 5TH FLOOR, BALTIMORE, MD, 21201, US

(73) Assignee: THE JOHNS HOPKINS UNIVERSITY, Baltimore, MD (US)

(21) Appl. No.: 12/811,604

(22) PCT Filed: Jan. 5, 2009
(86) PCT No.: PCT/US09/30104

§ 371 Date: Jul. 2, 2010

Related U.S. Application Data
(60) Provisional application No. 61/009,917, filed on Jan. 3, 2008.
(60) Provisional application No. 61/091,707, filed on Aug. 25, 2008.

(51) Int. Cl. A61K 39/395; A61K 38/02; A61P 35/00

(52) U.S. Cl. 424/174.1; 514/18.9

(57) ABSTRACT

Compositions and methods for restoring killing of cancer cells are provided. Preferably, the compositions are administered to a subject in an effective amount to antagonize, inhibit, reduce, or block B7-H1 mediated signal transduction in cancer cells expressing B7-H1. It has been discovered that B7-H1 transmits an anti-apoptotic signal in cancer cells that increases the resistance of the cancer cells to CTL mediated cytolysis and to Fas induced cell death. It is believed that blocking the transmission of the anti-apoptotic signal by B7-H1 increases the susceptibility of the cancer cells to apoptosis and CTL cytolysis thereby enhancing the death of cancer cells. Preferred compounds or B7-H1 antagonists include antibodies that bind to B7-H1, B7-H1 receptors, or ligands of B7-H1 such as PD-I or B7-1. Additional B7-H1 antagonists include small molecules, for example small molecules that bind the cytoplasmic portion of B7-H1 or the extracellular portion of B7-H1 and inhibit, reduce, block or interfere with B7-H1 signal transduction. Methods for treating one or more symptoms associated with cancer or hyperproliferation are also provided.

### GOVERNMENT SUPPORT

This invention was made with government support under grant numbers CA85721, CA97085 and CA113341 awarded by National Institutes of Health. The government has certain rights in the invention.

### FIELD OF THE INVENTION

The invention is generally related to methods and compositions for treating or preventing aberrant cellular proliferation.

### BACKGROUND OF THE INVENTION

Cancer has an enormous physiological and economic impact. For example a total of 1,437,180 new cancer cases and 565,650 deaths from cancer are projected to occur in the United States in 2008 (Jemal, A., Cancer J Clin, 58:71-96 (2008)). The National Institutes of Health estimate overall costs of cancer in 2007 at $219.2 billion: $89.0 billion for direct medical costs (total of all health expenditures); $18.2 billion for indirect morbidity costs (cost of lost productivity due to illness); and $112.0 billion for indirect mortality costs (cost of lost productivity due to premature death). Although many cancer therapies are available, they are usually associated with adverse side-effects. New treatments with fewer side effects are needed.

Cancer cells display altered surface molecular signatures that distinguish them quantitatively and qualitatively from their normal derivatives. These modifications in receptor and ligand expression commonly facilitate tumor growth and progression or to evasion of host defense mechanisms (Zitvogel, L., Nat Rev Immunol., 6:715-727 (2006); Friedl, P., et al., Nat Rev Cancer., 3:362-374 (2003)). For example, some tumor cells down-regulate their cell surface major histocompatibility complex (MHC) which is required for recognition by tumor antigen-specific T lymphocytes (Garrido, F. et al., Immunol Today, 14:491-499 (1993)). As a result, these tumor cells become less recognizable by the immune system and more resistant to immune-mediated destruction. Another example is that, during progression, cancer cells frequently over-express proteases and modify glycosylation of cell surface proteins that are normally involved in tissue repair, remodeling and homeostasis to facilitate invasion and metastasis (Liotta, L. A., et al. Cell, 64:327-336 1991); Hakomori, S., Cancer Res, 56:5309-5318 (1996)). In general, these modifications in cell membrane ligands and receptors regulate interactions between tumor cells and non-transformed cells in the microenvironment in a fashion that enhances tumor growth, invasion and immune resistance.

An immunoglobulin-like molecule termed B7-H1 is either constitutively or inducibly expressed by the majority of human and rodent cancer cells (Dong, H. et al., Nat. Med. 8:793-800 (2002); Strome, S. E. et al. Cancer Res. 63:6501-6505 (2003)). Ample evidence demonstrates that B7-H1 acts as a ligand for receptor programmed death-1 (PD-1), to deliver an inhibitory signal to T cells, leading to inhibition of immune responses (Chen, L., et al. Nat Rev Immunol, 4:336-347 (2004)). The mechanisms underlying B7-H1/PD-1 mediated suppression include induction of apoptosis, anergy, unresponsiveness and exhaustion of T cells (Dong, H., Nat. Med. 8:793-800 (2002)). Interaction between B7-H1 and PD-1 is also shown to participate in the suppression of autoimmune diseases and transplantation rejection in animal models (Dudler, J. et al., Transplantation, 82:1733-1737 (2006); Nishimura, H., et al. Immunity, 11:141-151 (1999); Okazaki, T., et al., Nat Med. 9:1477-1483 (2003); Latchman, Y. E., et al., Proc Natl Acad Sci USA., 101:10691-10696). A recent study suggests that B7-H1, in addition to PD-1, also binds 137-1 (CD80) on T cells to inhibit their activation (Butte, M. J., et al. Immunity, 27:111-122 (2007)). B7-H1+ tumor cells are much more resistant to CD8+ cytolytic T cells (CTL)-mediated destruction in vitro than their B7-H1 negative parental cells and this resistance is correlated with decreased efficacy of immunotherapy in mouse tumor models (Hirano, F., et al., Cancer Res., 65:1089-1096 (2005); Iwai, Y., et al., Proc Natl Acad Sci USA, 99:12293-12297 (2002); Blank, C., et al., Cancer Res, 64:1140-1145 (2004)). Ablation of B7-H1 and PD-1 interaction by neutralizing antibodies could restore CTL-mediated lysis of tumor cells in vitro, suggesting B7-H1/PD-1 interaction forms a barrier between tumor cells and CTLs and this phenomenon has been termed as “molecular shield” (Hirano, F., et al., Cancer Res., 65:1089-1096 (2005)). These results have been interpreted as inhibition of CTL activity induced by unidirectional engagement of PD-1 on the T cell by B7-H1 on the tumor cells. The exact mechanism of action remains unknown.

Therefore, it is an object of the invention to provide methods and compositions for treating or preventing aberrant cellular proliferation or signaling.

It is another object of the invention to provide methods and compositions for modulating B7-H1 signal transduction, preferably inhibiting B7-H1 signal transduction in cancer cells expressing B7-H1.

It is still another embodiment to provide compositions and methods for blocking counter-receptors (ligands) of B7-H1, including but not limited to B7-1 and PD-1.

### SUMMARY OF THE INVENTION

Compositions and methods for restoring killing of cancer cells are provided. Preferably, the compositions are administered to a subject in an effective amount to antagonize, inhibit, reduce, or block B7-H1 mediated signal transduction in cancer cells expressing B7-H1. It has been discovered that B7-H1 transmits an anti-apoptotic signal in cancer cells that increases the resistance of the cancer cells to CTL mediated cytolysis and to Fas induced cell death. It is believed that blocking the transmission of the anti-apoptotic signal by B7-H1 increases the susceptibility of the cancer cells to apoptosis and CTL cytolysis thereby enhancing the death of cancer cells. Preferred compounds or B7-H1 antagonists include antibodies that bind to B7-H1, B7-H1 receptors, or ligands of B7-H1. Representative ligands or counter-receptors include B7-1 and PD-1. Additional B7-H1 antagonists include small molecules, for example small molecules that bind the cytoplasmic portion of B7-H1 or the extracellular portion of B7-H1 and inhibit, reduce, block or interfere with B7-H1 signal transduction.

Pharmaceutical compositions are provided that include an effective amount of one or more B7-H1 antagonists to inhibit or reduce B7-H1-mediated cell signaling in cancer cells expressing B7-H1, wherein the inhibition of B7-H1-mediated cell signaling in the cancer cell promotes apoptosis of the cancer cell relative to a control.

Methods for increasing cancer cell death in a subject by administering to the subject an effective amount of a B7-H1 antagonist to inhibit B7-H1-mediated signaling in cancer cells expressing B7-H1 are also provided. Additionally, methods for promoting apoptosis or increasing susceptibility to apoptosis or CTL mediated cell death in cancer cells expressing B7-H1 by contacting the cancer cells with an effective amount of a B7-H1 antagonist to interfere, inhibit or reduce B7-H1-mediated signal transduction in the cancer cells are described.

Finally, methods for screening for and selecting modulators of B7-H1-mediated signaling are provided.

### BRIEF DESCRIPTION OF THE DRAWINGS

FIG. 1A is a line graph of percent lysis versus Effector/Target (BIT) ratio of Mock/P815:CTL mAB (◯), B7-H1/P815:CTL mAb (□), Mock/P815:10B5 (), and B7-H1/P815:10B5 (▪). Each point is the means of triplicates with standard deviation (SD). The data is representative of three experiments. FIG. 1B is a line graph of percent lysis versus E/T ratio of activated 2C CTLs: 51-Cr-labeled Renca cells treated with control IgG (◯)) or anti-murine B7-H1 antibody (clone 10B5) (▪) for 4 hrs. Each point is the means of triplicates with standard deviation (SD). The data is representative of three experiments.

FIG. 2A is a schematic of wild type PD-1 (PD-1) and intracellular domain-truncated PD-1 (ΔPD-1) genes. IgV domain; IgV, TM; transmembrane domain, CY; cytoplasmic domain, GFP; gene encoding for green fluorescence protein. FIG. 2B are flow cytometry histograms of the expression of PD-1 on 2C×PD-1KO T cells upon transduction using anti-mouse PD-1 mAb (G4). FIGS. 2C and D are a line graphs of percent lysis versus E/T ratio of activated PD-1/PD-1⁰2C (◯), ΔPD-1/PD-1⁰2C (Δ) or non-transduced PD-1⁰2C (Δ) to ⁵¹Cr-labeled mock/P815 (FIG. 2C) or B7-H1/P815 (FIG. 2D) (solid symbols) cells in the presence of control IgG or anti-mouse B7-H1 mAb (10B5) for 4 hr. Each point is the means of triplicates with standard deviation (SD). The data is representative of three experiments.

FIG. 3A is a schematic of the wild type B7-H1 (B7-H1) and cytoplasmic domain-truncated B7-H1 (ΔB7-H1) genes. IgV domain; IgV, IgC domain; IgC. TM; transmembrane domain, CY; cytoplasmic domain. GFP; gene encoding green fluorescence protein. FIGS. 3B-G are flow cytometry histograms showing the expression of wild type B7-H1 (B7-H1) (3B) or truncated B7-H1 (ΔB7-H1) (3C) on P815 cells after transfection using anti-mouse B7-H1 mAb (10B5). FIGS. 3D and 3E are flow cytometry histograms showing the expression of wild type B7-H1 (B7-H1) (3D) or truncated B7-H1 (ΔB7-H1) (3E) on P815 cells after transfection using murine PD-1Ig fusion protein. FIGS. 3F and 3G are flow cytometry histograms showing the expression of wild type B7-H1 (B7-H1) (3F) or truncated B7-H1 (ΔB7-H1) (3G) on P815 cells after transfection using anti-H-2L^{d} mAb. FIG. 3H is a line graph of percent lysis versus E/T ratio of activated 2C CTLs incubated at indicated E/T ratios with ⁵¹Cr-labeled mock/P815 (◯), B7-H1/P815 (□) or ΔB7-H1/P815 (Δ) cells in the presence of control (Ctl) IgG (open symbols) or anti-mouse B7-H1 mAb (10135) (solid symbols) for 4 hrs. Each point is the means of triplicates with standard deviation (SD). The data is representative of at least three experiments.

FIG. 4A is a schematic of the full length B7-H1 (B7-H1), full length B7-DC (B7-DC) and the chimera of B7-H1 and B7-DC (B7-DC/H1) genes. IgV domain; IgV, IgC domain; IgC. TM; transmembrane domain, CY; cytoplasmic domain. FIG. 4B is a flow cytometry histogram showing the expression of B7-DC on B7-DC/P815 using anti-mouse B7-DC mAb (clone TY25). FIG. 4C s a flow cytometry histogram showing the expression of B7-DC on B7-DC/H1/P815 using anti-mouse B7-DC mAb (clone TY25). FIG. 4D is a flow cytometry histogram showing the ability of B7-DC/P815 to bind PD-1Ig fusion protein. FIG. 4E is a flow cytometry histogram showing the ability of B7-DC/H1/P815 to bind PD-1Ig fusion protein. FIG. 4F is a line graph of percent lysis versus E/T ratio or activated 2C CTLs incubated at indicated E/T ratios of ⁵¹Cr-labeled mock/P815 (◯), full length B7-DC/P815 (□) or B7-DC/H1/P815 (Δ) cells in the presence of control IgG or anti-mouse B7-DC mAb (shaded symbols) for 4 hr. Each point is the means of triplicates with standard deviation (SD). The data is representative of at least three experiments.

FIG. 5 is a line graph of mean tumor diameters (mm) versus days after tumor inoculation for groups of 10 DBA/2 mice given s.c. injections of 5×10⁴ mock/P815 (◯), B7-H1/P815 (□) or ΔB7-H1/P815 cells (Δ) on day 0. Mice were then treated i.p. with 200 μg control IgG or anti-mouse CD137 mAb (clone 2A, shaded symbols) at days 7 and 10. Each point is the means of 10 with standard deviation (SD). The data is representative of at least three experiments.

FIG. 6A is a line graph of percent lysis versus E/T ratio of activated PD-1⁰2C CTLs to B7-H1/P815 in wells pre-coated with either control IgG (Ctl Ig) (◯) or murine PD-1Ig (PD-1Ig) (▪) at 5 μg/ml for 18 hrs. After extensive washing, cells were labeled with ⁵¹Cr and further incubated with at indicated E/T ratios for 4 hrs. CTL activity was determined in a ⁵¹Cr release assay. Each point is the means of triplicates with standard deviation (SD). The data is representative of at least three experiments. FIGS. 68 and 6C are flow cytometry histograms of Fas expression on mock/P815 (FIG. 6B) or B7-H1/P815 (FIG. 6C) cells before and after culture with immobilized murine PD-1Ig using anti-murine Fas mAb.

### DETAILED DESCRIPTION OF THE INVENTION

### I. Definitions

Unless otherwise defined, all technical and scientific terms used herein have the same meaning as commonly understood by one of ordinary skill in the art to which this invention pertains. All publications, patent applications, patents, and other references mentioned herein are incorporated by reference in their entirety. In case of conflict, the present specification, including definitions, will control. In addition, the materials, methods, and examples are illustrative only and not intended to be limiting.

The term “effective amount” or “therapeutically effective amount” means a dosage sufficient to reduce, prevent, or inhibit one or more symptoms associated with cancer or to otherwise provide a desired pharmacologic and/or physiologic effect. The precise dosage will vary according to a variety of factors such as subject-dependent variables (e.g., age, immune system health, etc.), the disease, and the treatment being effected.

A “fragment” of a B7-H1 polypeptide is a fragment of the polypeptide that is shorter than the full-length polypeptide. Generally, fragments will be five or more amino acids in length. An antigenic fragment has the ability to be recognized and bound by an antibody.

The terms “individual,” “individual,” “subject,” and “patient” are used interchangeably herein, and refer to a mammal, including, but not limited to, rodents, simians, humans, mammalian farm animals, mammalian sport animals, and mammalian pets.

As used herein, “operably linked” with regard to nucleic acids means incorporated into a genetic construct so that expression control sequences effectively control expression of a coding sequence of interest.

The terms “polypeptide” and “protein” are used interchangeably and mean any peptide-linked chain of amino acids, regardless of length or post-translational modification. Embodiments include B7-H1 polypeptides with conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

The term “B7-H1 antagonists” refers to a protein, lipid, or nucleic acid, peptidomimetics, small molecule, or carbohydrate that interferes with, inhibits, or blocks one or more biological activities of B7-H1. In a preferred embodiment, the B7-H1 antagonists interfere with B7-H1 mediated signaling in cancer cells expressing B7-H1.

### II. B7-H1 Antagonists

It has been discovered that B7-H1 can act as a receptor to transmit an anti-apoptotic signal to cancer cells, leading to resistance to cytolysis by CTLs as well as Fas and drug-induced apoptosis. In addition, elimination of the intracellular domain of B7-H1 can ablate cancer resistance to immune destruction, which is accompanied with regression of tumors in mouse models. One embodiment provides methods and compositions for modulating B7-H1 mediated signal transduction. Preferred methods and compositions increase or promote cell death or apoptosis of cells expressing B7-H1 or increase the susceptibility to apoptosis or CTL mediated cell death. Preferred cells expressing B7-H1 include but are not limited to cancer cells. The disclosed compositions antagonize B7-H1 signal transduction in cancer cells and inhibit or reduce B7-H1 signal transduction relative to a control. B7-H1 antagonists can be polypeptide antagonists including an antibody or antigen binding fragment thereof specific for B7-H1 or a molecule that binds to B7-H1, for example B7-1 or PD-1, and interferes with B7-H1 signaling in cancer cells. Other B7-H1 antagonists are non-protein, small molecule antagonists. Small molecule antagonists are typically less than 1,000 daltons.

A. B7-H1

A number of prior studies indicate that cells expressing B7-H1 can evade immune destruction by engaging inhibitory receptor(s) on T cells (Hirano, F. et al., Cancer Res., 65:1089-1096 (2005); Iwai, Y. et al., Proc Natl Acad Sci USA, 99:12293-12297 (2002); Blank, C. et al., Cancer Res., 64:1140-1145 (2004)). These studies are particularly intriguing because upregulation of B7-H1 is found in the majority of human cancers. Prior studies, however, focused on the function of B7-H1 as a ligand, i.e., upon interaction with the PD-1 receptor on T cells, an inhibitory signal would be delivered to T cells, leading to apoptosis, suppression, anergy and exhaustion (Dong, H. et al., Nat. Med., 8:793-800 (2002); Barber, D. L., Nature., 439:682-687 (2006); Freeman, G. J., et al., J Exp Med. 192:1027-1034 (2000); Tsushima, F., et al. Blood., 110:180-185 (2007); Goldberg, M. V., et al., Blood; 110:186-192 (2007)). This immune inhibitory function occurs during the induction of T cell activation in lymphoid organs and/or in the effector phase after migration of activated T cells to peripheral organs (Dudler, J. Transplantation, 82:1733-1737 (2006); Blank, C., et al. Cancer Res. 64:1140-1145 (2004)).

It has been discovered that B7-H1 inhibits anti-tumor immune responses by transmitting an anti-apoptotic signal to tumor cells. PD-1 signaling does not seem to suppress cytolytic function of CTLs in vitro. It is possible that PD-1-mediated dysfunction of CTL may require more than 4-6 hr exposure, which, however, is sufficient for B7-H1 to deliver anti-apoptotic function on tumor cells. Although it appears that the predominant mechanism in vitro appears to be B7-H1-mediated tumor resistance, both B7-H1-mediated tumor resistance and PD-1-mediated T cell dysfunction could play roles simultaneously in vivo. For example, immunotherapy of established tumors induced by ΔB7-H1/P815 was only partially active in comparison with tumors induced by wild type B7-1/P815 (FIG. 5), suggesting a role of PD-1-mediated immune dysfunction in vivo.

B7-H1 has multiple ligands or counter-receptors. One of the ligands, PD-1, is found to be expressed on T cells, B cells and a lymphoid/myeloid progenitor cell line (Ishida, Y. et al., Embo J., 11:3887-3895 (1992); Agata, Y., Int Immunol., 8:765-772 (1996)). A recent study shows that, in addition to PD-1, B7-H1 could also bind CD80 which is found in the majority of hematopoietic cells as well as stromal cells (Butte, M. J., et al., Immunity, 27:111-122 (2007)). Because immune cells including T and B cells are often found to infiltrate tumors, the data suggest a new mechanism by which cancer cells could utilize receptors of the immune system to escape from destruction even by non-immunologic agents.

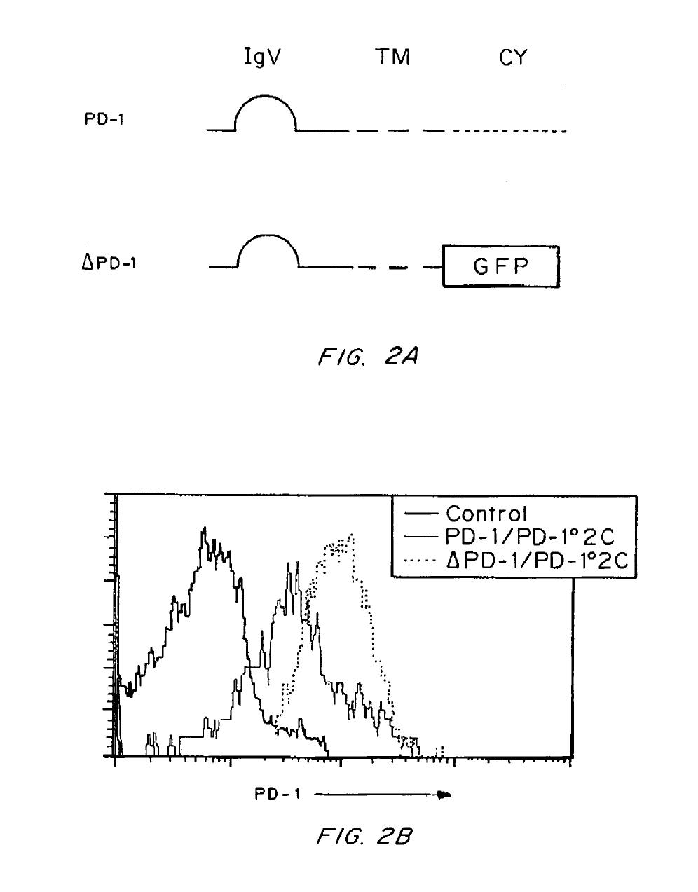

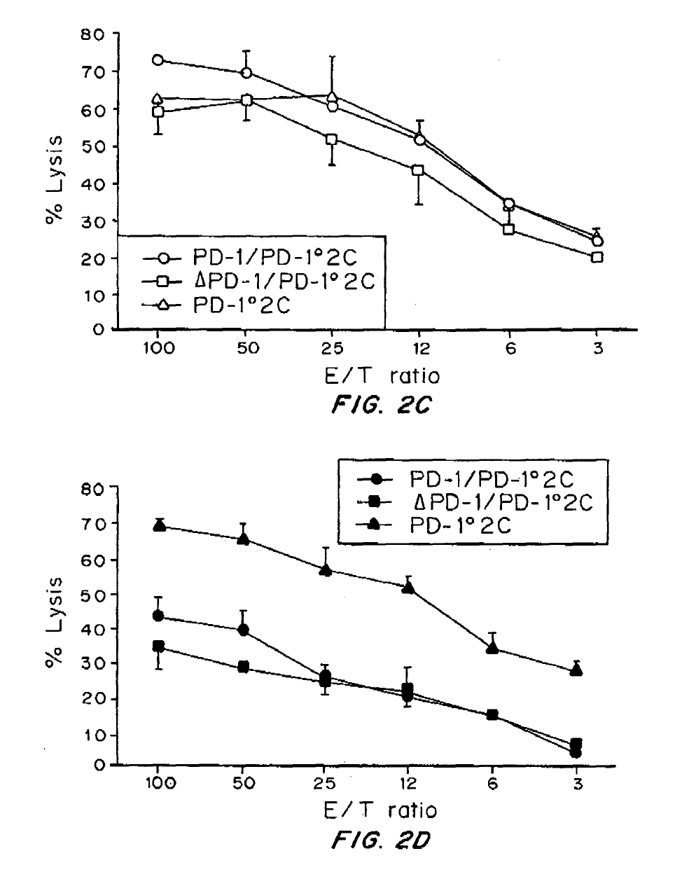

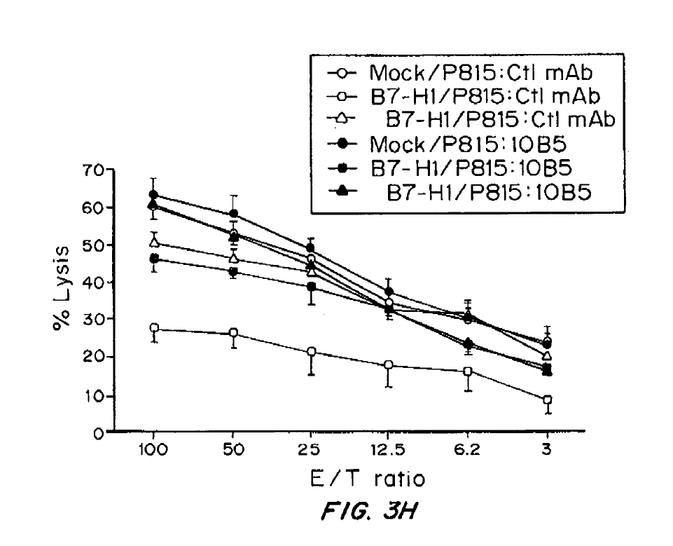

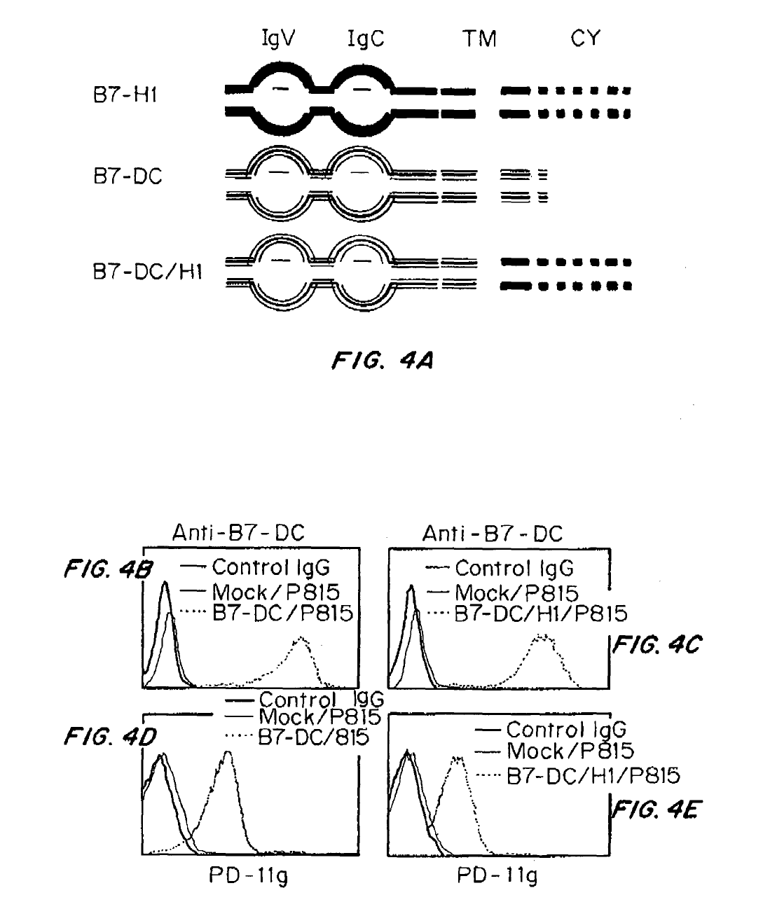

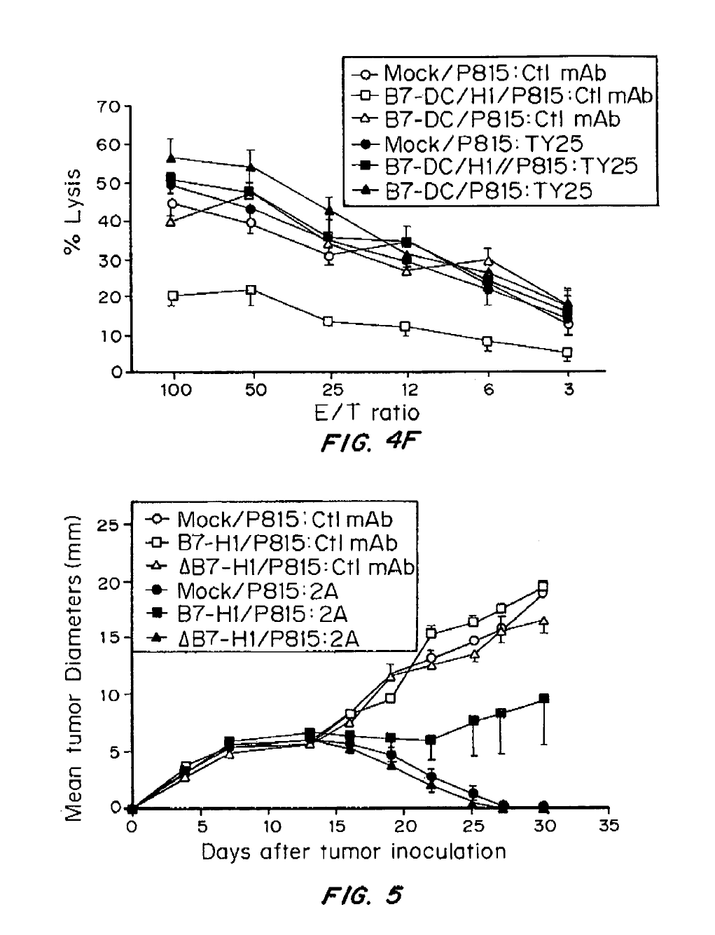

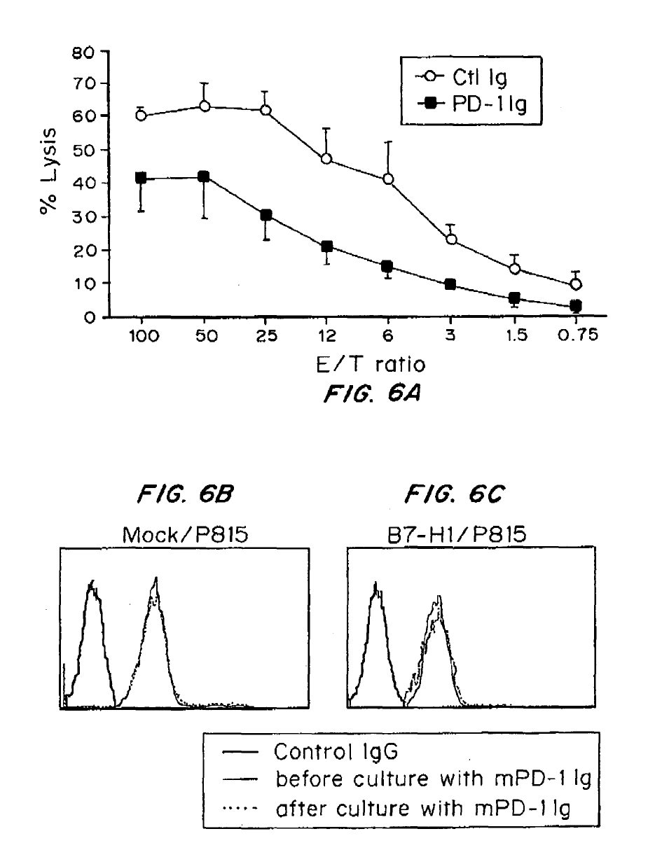

The intracellular domain of B7-H1 has 30 amino acids and does not contain any obvious motifs for signaling to known anti-apoptotic molecules. One possible explanation is that intracellular domain of B7-H1 binds adaptor molecules which deliver anti-apoptotic signal. By preparation of truncated B7-H1, the data provided herein demonstrates that intracellular domain of B7-H1 on cancer cells is required for the formation of the molecular shield. In contrast, the expression of truncated PD-1 or wild type PD-1 into PD-1−/−2C transgenic T cells confers CTL resistance in comparison with non-transduced PD-1−/−2C transgenic T cells (FIG. 2D). An alternative interpretation is that transduced PD-1 may interfere the TCR-MHC interaction. This, however, is considered unlikely because PD-1−/−2C transgenic T cells that express truncated or wild type PD-1 could lyse B7-H1 negative P815 equally well (FIG. 2C). Taken together, the data indicate that PD-1 acts as a ligand in this system to stimulate B7-H1. This notion is further supported by the experiments showing B7-DC, which is capable of binding to PD-1, on tumor cells does not induce resistance of tumor cells to CTL lysis while replacement of its intracellular domain with that from B7-H1 does (FIG. 4C). By comparison with B7-H1, murine B7-DC has a very short cytoplasmic tail (4 amino acids) and would not be expected to signal equivalently to B7-H1. Finally, incubation of B7-H1+ tumor cells in the presence of immobilized PD-1 is sufficient to induce resistance to apoptosis (FIG. 6). It has been shown that binding to B7-H1 to PD-1 induces an inhibitory signal toward T cells. It is likely that the molecular shield occurs very quickly within a couple of hours after exposure whereas induction of T cell suppression through PD-1 as a receptor requires more time. This is supported by a previous study showing that induction of apoptosis of human tumor antigen-specific T cells by B7-H1+ cancer cells required 2-3 days (Dong, H., et al. Nat. Med., 8:793-800 (2002)).

While the data demonstrate a role of B7-H1 in the induction of anti-apoptotic mechanism on cancer cells, the underlying biochemical pathways remain to be defined. Several major anti-apoptotic and apoptotic pathways were investigated using the Apoptosis Oligo GEArray® (SuperArray, Frederick, Md.) and no major differences were found between control Ig vs. PD-1Ig treatment on B7-H1/P815 cells in the expression of apoptosis related genes including TNF receptor family, Bcl-2 family, caspase family, IAP family, TRAF family, CARD family, death domain family, death effector domain family, CIDE family and anti-apoptosis genes etc. It has been shown that CTL-mediated lysis of target cells is mediated by at least two mechanisms: granule-mediated cytolysis and membrane receptor-mediated apoptosis. After contact of target cells, perforin could be released from CTL to form channels on target cells, which allows passage of important molecules such as granzyme B. Granzyme B will activate caspase 3 and/or cause mitochondrial disruption after cleavage of Bid (Pipkin, M. E., et al., Curr Opin Immunol. 19:301-308 (2007)), leading to apoptosis. In addition, cell surface apoptotic receptors such as Fas or TRAIL may also induce apoptosis of target cells after engagement their ligands or counter-receptor (Peter, M. E., Curr Opin Immunol., 10:545-551 (1998); Smyth, M. J., et al., Immunity, 18:1-6 (2003)). B7-H1 appears to have a broad role in the resistance of apoptosis of tumor cells, including CTL-mediated death as well as Fas- and Staurosporine-mediated apoptosis. It is thus possible that B7-H1 represents an early signal in the cascade to induce the inhibition of both pathways. In the context of broad expression of B7-H1 on tumor cells, this mode of anti-apoptosis may play a significant role in the prevention of tumor cell death by immune therapy and chemotherapy.

B. Polypeptide Antagonists of B7-H1

1. Polypeptide Antagonists that Block B7-H1 Counter-Receptors

PD-1 and B7-1 are expressed on T cells and can stimulate B7-H1 expressed on tumor cells and cause an anti-apoptotic signal to be transmitted into the tumor cell. One embodiment provides compositions that bind to PD-1, B7-1 or a combination thereof and inhibit or prevent PD-1 and B7-1 on T cells from binding to B7-H1 expressed on tumor cells. Preferred compositions include polypeptides that bind to PD-1, B7-1 or a combination thereof and optionally sterically interfere with binding to B7-H1. Preferred polypeptides include antibodies or antigen-binding fragments thereof, peptidomimetics, and fusion proteins. One embodiment provides B7-DC fusion proteins or variants thereof that bind to PD-1 and antagonize B7-H1 by interfering or blocking the interaction between PD-1 and B7-H1. Preferably, the B7-DC fusion proteins do not trigger signal transduction through PD-1 into cells expressing PD-1.

2. Antibody Antagonists of B7-H1

Antibodies that bind to B7-H1 or to a B7-H1 receptor or ligand and inhibit or reduce B7-H1 signal transduction are provided. In one embodiment, the B7-H1 antagonist is an antibody that binds to at least two different B7-H1 receptors. Because B7-H1 has more than one receptor, blockage of a multiple receptors may increase the amount of inhibition of B7-H1 signal transduction. The antibody can be polyclonal, monoclonal, chimeric, humanized or a fragment of the antibody that binds to B7-H1 or a receptor or ligand thereof. Two or more antibodies can be used to inhibit B7-H1 signal transduction. For example an anti-B7-H1 antibody can be used together with an anti-PD-1 antibody or an anti-B7-1 antibody.

Methods of producing antibodies are well known and within the ability of one of ordinary skill in the art.

The antibodies disclosed herein specifically bind to a B7-H1, B7-H1 receptor, or B7-H1 ligand such as B7-1 or PD-1 and are capable of reducing or inhibiting the binding of B7-H1. These antibodies are defined as “blocking”, “function-blocking” or “antagonistic” antibodies. In preferred embodiments the antagonistic antibodies specifically bind to a portion of the extracellular domain of B7-H1.

The immunogen used to generate the antibody may be any immunogenic portion of B7-H1 or B7-H1 receptor. Preferred immunogens include all or a part of the extracellular domain of human B7-H1, where these residues contain the post-translation modifications, such as glycosylation, found on native B7-H1 or B7-H1 receptor. Immunogens including the extracellular domain or immunogenic fragments thereof are produced in a variety of ways known in the art, e.g., expression of cloned genes using conventional recombinant methods, isolation from cells of origin, cell populations expressing high levels of B7-H1 or B7-H1 receptor.

The antibodies disclosed herein are capable of binding to a B7-H1, B7-H1 receptor, or a B7-H1 ligand polypeptide having at least about 70%, more preferably 75%, 80%, 85%, 90%, 95% identity to human B7-H1, GENBANK Accession Number AAP37283.

The antibodies may be xenogeneic, allogeneic, syngeneic, or modified forms thereof, such as humanized or chimeric antibodies. The antibodies may also be anti-idiotypic antibodies. Antibodies, as used herein, also includes antibody fragments including Fab and F(ab)₂ fragments, and antibodies produced as a single chain antibody or scFv instead of the normal multimeric structure. The antibodies may be an IgG such as IgG1, IgG2, IgG3 or IgG4; or IgM, IgA, IgE or IgD isotype. The constant domain of the antibody heavy chain maybe selected depending on the effector function desired. The light chain constant domain may be a kappa or lambda constant domain.

3. Soluble Forms of B7-H1 Counter-Receptors

Additional forms of polypeptide B7-H1 antagonists include soluble forms of the B7-H1 counter-receptors B7-1 and PD-1. Soluble forms of B7-1 and PD-1 include the extracellular domain of B7-1 and PD-1 or a fragment thereof that binds B7-H1 on tumor cells but does not trigger signal transduction through B7-H1 into the tumor cell. The nucleotide and amino acid sequences for human B7-1 are provided by GENBANK accession no. NM_{—}005191. The nucleotide and amino acid sequences for human PD-1 are provided by GENBANK accession no. NOP_{—}005009.2

The amino acid sequence for the extracellular domain of human B7-1 is:

(SEQ ID NO: 15)

| | |
|---|---|
| MGHTRRQGTS PSKCPYLNFF QLLVLAGLSH FCSGVIHVTK | 60 |
| EVKEVATLSC GHNVSVEELA | |
| QTRIYWQKEK KMVLTMMSGD MNIWPEYKNR TIFDITNNLS | 120 |
| IVILALRPSD EGTYECVVLK | |
| YEKDAFKREH LAEVTLSVKA DFPTPSISDF EIPTSNIRRI | 180 |
| ICSTSGGFPE PHLSWLENGE | |
| ELNAINTTVS QDPETELYAV SSKLDFNMTT NHSFMCLIKY | 240 |
| GHLRVNQTFN WNTTKQEHFP | |
| DNL | 243 |

The amino acid sequence for the extracellular domain of PD-1 is:

(SEQ ID NO: 16)

| | |
|---|---|
| PGWFLDSPDR PWNPPTFSPA LLVVTEGDNA TFTCSFSNTS | 60 |
| ESFVLNWYRM SPSNQTDKLA | |
| AFPEDRSQPG QDCRFRVTQL PNGRDFHMSV VRARRNDSGT | 120 |
| YLCGAISLAP KAQIKESLRA | |
| ELRVTERRAE VPTAHPSPSP RPAGQFQTLV, | 150 |
| or amino acids 21-170 of | |
| GENBANK accession no. NP_005009.2. | |

4. Fusion Proteins

Fusion proteins can also be produced that bind to B7-H1 on tumor cells and prevent signal transduction through B7-H1 into the tumor cells. Alternatively, fusion proteins can be designed to bind to B7-H1 counter receptors and prevent the counter receptors from interacting with B7-H1 on tumor cells. The provided fusion polypeptides have a first fusion partner having all or a part of a B7-H1 counter-receptor extracellular domain fused (i) directly to a second polypeptide or, (ii) optionally, fused to a linker peptide sequence that is fused to the second polypeptide. Suitable counter-receptor extracellular domains include but are not limited to those from B7-1 and PD-1. The presence of the fusion partner can alter the solubility, affinity and/or valency of the fusion protein. As used herein, “valency” refers to the number of binding sites available per molecule. The disclosed fusion proteins include any combination of amino acid alteration (i.e., substitution, deletion or insertion), fragment of a B7-H1 counter-receptor extracellular domain.

The second polypeptide binding partner may be N-terminal or C-terminal relative to the first polypeptide binding partner. In a preferred embodiment, the second polypeptide is C-terminal to the first polypeptide binding partner.

A large number of polypeptide sequences that are routinely used as fusion protein binding partners are well known in the art. Examples of useful polypeptide binding partners include, but are not limited to, green fluorescent protein (GFP), glutathione S-transferase (GST), polyhistidine, myc, hemaglutinin, Flag™ tag (Kodak, New Haven, Conn.), maltose E binding protein and protein A.

One embodiment provides a variant B7-DC fusion protein including the extracellular domain of B7-DC or fragment thereof modified or mutated as needed to reduce or eliminate signal transduction through PD-1 upon binding PD-1 fused to one or more domains of an Ig heavy chain constant region, preferably having an amino acid sequence corresponding to the hinge, C_{H2} and C_{H3} regions of a human immunoglobulin Cγ1 chain. Preferred B7-DC variants have one or more amino acid substitutions, deletions, or insertions that inhibit or reduce signal transduction through PD-1 when the B7-DC variant binds to PD-1. It will be appreciated that the one or more amino acid substitutions, deletions, or insertions are in the extracellular domain of the B7-DC variant. The B7-DC fusion protein antagonizes B7-H1 by binding to PD-1 and interfering or blocking the interaction between PD-1 and B7-H1. The constant region preferably includes a mutation for example N297Q to eliminate or reduce Fc receptor binding.

Still another embodiment provides a tetramer construct having a BirA substrate fused to the extracellular domain of a variant B7-DC polypeptide. Methods for making tetramer constructs are known in the art (see Pertovas, et al., J. Exp. Med., 203:2281 (2006)). The B7-DC polypeptide tetramer construct antagonizes B7-H1 by binding to PD-1 and interfering or blocking with the interaction between PD-1 and B7-H1. Preferably, the tetramer does not trigger signal transduction through PD-1 into cells expressing PD-1.

As noted above, fusion proteins that bind to PD-1 and B7-1 can be produced such that the fusion proteins bind to PD-1 or B7-1 but do not cause a signal to be transmitted through PD-1 or B7-1 into the T cell. For example, the extracellular domain of B7-H1 or a fragment of B7-H1 that can bind to PD-1 or B7-1 can be fused to a second polypeptide. Fragments of B7-H1 that can bind to PD-1, B7-1 or both include fragments containing all or part of the extracellular domain of B7-H1. Some fragments may contain all or part of the extracellular domain as wells as at least one amino acid from the transmembrane domain.

C. Peptidomimetic B7-H1 Antagonists

Peptidomimetics that antagonize B7-H1 signal transduction in cancer cells are also provided. Peptidomimetics are compounds which mimic the biological activity of peptides while offering the advantages of increased bioavailability, biostability, bioefficiency, and bioselectivity against the natural biological target of the parent peptide. Peptidomimetics have general features analogous to their parent structures, polypeptides, such as amphiphilicity. Examples of such peptidomimetic materials are described in Moore et al., Chem. Rev. 101(12), 3893-4012 (2001). As used herein, the term “peptidomimetic” includes chemically modified peptides and peptide-like molecules that contain non-naturally occurring amino acids, peptoids, and the like. Preferred substituents in peptidomimetic B7-H1 antagonists include those which correspond to the backbone or side chains of naturally occurring B7-H1 polypeptides with high affinity for the receptor. Suitable classes of eptidomimetics include, but are not limited to peptoids, retro-inverso peptides, azapeptides, urea-peptidomimetics, sulphonamide peptides/peptoids, oligoureas, oligocarbamates, N,N′-linked oligoureas, oligopyrrolinones, oxazolidin-2-ones, azatides, and hydrazino peptides.

D. Small Molecule B7-H1 Antagonists

Additional B7-H1 antagonists include small molecule antagonists. The term “small molecule” refers to compounds having a molecular weight of less than about 1,000 Daltons and are non-polypeptide or non-nucleic acid molecules. Small molecule B7-H1 antagonists can be obtained by screening libraries of molecules, for example combinatorial libraries of organic compounds, for binding to B7-H1. Alternatively, small molecule B7-H1 antagonists can be designed based on the X-ray crystallographic structure of B7-H1. Preferred small molecule B7-H1 antagonists bind to the intracellular cytoplasmic tail of B7-H1 and interfere or inhibit B7-H1 signal transduction.

E. Combination Therapy

1. Chemotherapeutic Agents

The B7-H1 antagonist can also be combined with a one or more additional therapeutic agents. Representative therapeutic agents include, but are not limited to chemotherapeutic agents and pro-apoptotic agents. Representative chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2) and combinations thereof.

In certain embodiments, more than one B7-H1 antagonist can be used in combination to increase or enhance an immune response in a subject.

2. Costimulatory Molecule Fusion Proteins

In other embodiments, B7-H1 antagonist may be co-administered with compositions containing B7 family costimulatory molecules. The B7 costimulatory polypeptide may be of any species of origin. In one embodiment, the costimulatory polypeptide is from a mammalian species. In a preferred embodiment, the costimulatory polypeptide is of murine or human origin. Useful human B7 costimulatory polypeptides have at least about 80, 85, 90, 95 or 100% sequence identity to the B7-DC polypeptide encoded by the nucleic acid having GenBank Accession Number NM_{—}025239; the B7-1 polypeptide encoded by the nucleic acid having GenBank Accession Number NM_{—}005191; the B7-2 polypeptide encoded by the nucleic acid having GenBank Accession Number U04343 or; the B7-H5 polypeptide encoded by the nucleic acid having GenBank Accession Number NP_{—}071436. B7-H5 is also disclosed in PCT Publication No. WO 2006/012232.

In a preferred embodiment, the B7 family costimulatory molecules are provided as soluble fusion proteins. Soluble fusion proteins of B7 costimulatory molecules that form dimers or multimers and have the ability to crosslink their cognate costimulatory receptors and thereby function as receptor agonists. B7 family costimulatory fusion polypeptides disclosed herein have a first fusion partner including all or a part of a costimulatory B7 protein fused (i) directly to a second polypeptide or, (ii) optionally, fused to a linker peptide sequence that is fused to the second polypeptide. The fusion proteins can include full-length B7-costimulatory polypeptides, or can contain a fragment of a full length B7 costimulatory polypeptides

In one embodiment, the first fusion partner is a fragment of a B7 family costimulatory molecule, including, but not limited to B7-DC, B7-1, B7-2, or B7-H5. As used herein, a fragment of B7 costimulatory molecule refers to any subset of the polypeptide that is a shorter polypeptide of the full length protein. Useful fragments are those that retain the ability to bind to their natural ligands. A B7 costimulatory polypeptide that is a fragment of full-length B7 costimulatory molecule typically has at least 20 percent, 30 percent, 40 percent, 50 percent, 60 percent, 70 percent, 80 percent, 90 percent, 95 percent, 98 percent, 99 percent, 100 percent, or even more than 100 percent of the ability to bind its natural ligand(s) as compared to full-length B7 costimulatory molecules.

Fragments of B7 costimulatory polypeptides include soluble fragments. Soluble B7 costimulatory polypeptide fragments are fragments of B7 costimulatory polypeptides that may be shed, secreted or otherwise extracted from the producing cells. Soluble fragments of B7 costimulatory polypeptides include some or all of the extracellular domain of the receptor polypeptide, and lack some or all of the intracellular and/or transmembrane domains. In one embodiment, B7 costimulatory polypeptide fragments include the entire extracellular domain of the B7 costimulatory polypeptide. In other embodiments, the soluble fragments of B7 costimulatory polypeptides include fragments of the extracellular domain that retain B7 costimulatory biological activity. It will be appreciated that the extracellular domain can include 1, 2, 3, 4, or 5 amino acids from the transmembrane domain. Alternatively, the extracellular domain can have 1, 2, 3, 4, or 5 amino acids removed from the C-terminus, N-terminus, or both.

Generally, the B7 costimulatory polypeptides or fragments thereof are expressed from nucleic acids that include sequences that encode a signal sequence. The signal sequence is generally cleaved from the immature polypeptide to produce the mature polypeptide lacking the signal sequence. It will be appreciated that the signal sequence of B7 costimulatory polypeptides can be replaced by the signal sequence of another polypeptide using standard molecule biology techniques to affect the expression levels, secretion, solubility, or other property of the polypeptide. The signal sequence that is used to replace the signal sequence can be any known in the art.

B7 costimulatory molecule fusion polypeptides include variant polypeptides that are mutated to contain a deletion, substitution, insertion, or rearrangement of one or more amino acids relative to the wild-type polypeptide sequence. Useful variant B7 costimulatory fusion proteins are those that retain the ability to bind to costimulatory receptor polypeptides. Variant B7 costimulatory fusion polypeptides typically have at least 20 percent, 30 percent, 40 percent, 50 percent, 60 percent, 70 percent, 80 percent, 90 percent, 95 percent, 98 percent, 99 percent, 100 percent, or even more than 100 percent of the ability to bind to B7 costimulatory receptor polypeptides as compared to full-length B7 costimulatory molecules,

Variant B7-H5 fusion polypeptides can have any combination of amino acid substitutions, deletions or insertions. Variant polypeptides may contain one or more amino acid deletions, substitutions, insertions, or rearrangements within either or all of the first fusion partner, the second polypeptide, and/or the optional linker peptide sequence.

3. Adoptive Transfer

Adoptive T-cell therapy is a promising strategy for the treatment of patients with established tumors but is often limited to specific cancers where tumor-infiltrating lymphocytes, the source of T cells for ex vivo culture, can be obtained. One embodiment provides a method for treating cancer by administering an effective amount of an antagonist for B7-H1 to inhibit or reduce B7-H1 mediated signal transduction in a tumor cell in combination with adoptive 1-cell therapy of antigen specific T cells. The adoptive 1-cell transfer can be administered to the subject prior to or following administration of the antagonist of B7-H1.

Antigen-specific 1-cell lines can be generated by in vitro stimulation with antigen followed by nonspecific expansion on CD3/CD28 beads. The ability to expand antigen-specific T cells can be assessed using IFN-gamma and granzyme B enzyme-linked immunosorbent spot. The phenotype of the resultant 1-cell lines can be evaluated by flow cytometry, including the presence of FOXP3-expressing CD4(+) T cells. Amplification of antigen-specific T cell populations from Peripheral Blood Mononuclear Cells (PBMCs) is usually performed through repeated in-vitro stimulation with optimal length antigenic peptides in the presence of IL-2. Low doses of IL-2 (between 10 and 50 U/ml) have been used traditionally to avoid the activation/expansion of lymphokine-activated killer cells, as revealed in chromium release assays that were commonly employed to monitor specific T cell expansion. Concentrations of antigenic peptides can be 0.1-10 μM.

a. Tumor-Specific and Tumor-Associated Antigens

Antigens useful for expanding T cells can be obtained from biopsies of tumors from the subject to be treated. The antigens can be biochemically purified from the tumor biopsy. Alternatively, the antigens can be recombinant polypeptides. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.) (see, e.g., U.S. Pat. No. 6,699,475; Jager, et al., Int. J. Cancer, 106:817-20 (2003); Kennedy, et al., Int. Rev. Immuno, 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, and because of this, these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309:883 (1983); Lloyd, et al., Int. J. Canc., 71:842 (1997). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (FLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17(4B):2945 (1997); Kudoh, et al., Gynecol. Obstet. Invest., 47:52 (1999)). Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen, mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies.

A tumor antigen may include a cell surface molecule. Tumor antigens of known structure and having a known or described function, include the following cell surface receptors: HER1 (GenBank Accession No. U48722), HER2 (Yoshino, et al., J. Immunol., 152:2393 (1994); Disis, et al., Canc. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Ace. Nos. L07868 and T64105), epidermal growth factor receptor (EGFR) (GenBank Ace. Nos. U48722, and K03193), vascular endothelial cell growth factor (GenBank No. M32977), vascular endothelial cell growth factor receptor (GenBank Ace. Nos. AF022375, 1680143, U48801 and X62568), insulin-like growth factor-I (GenBank Ace. Nos. X00173, X56774, X56773, X06043, European Patent No. GB 2241703), insulin-like growth factor-II (GenBank Ace. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Ace. Nos. M38651, X03635, X99101, U47678 and M12674), progesterone receptor (GenBank Ace. Nos. X51730, X69068 and M15716), follicle stimulating hormone receptor (FSH-R) (GenBank Acc. Nos. Z34260 and M65085), retinoic acid receptor (GenBank Ace. Nos. L12060, M60909, X77664, X57280, X07282 and X06538), MUC-1 (Barnes, et al., Proc. Nat. Acad. Sci. USA, 86:7159 (1989); GenBank Ace. Nos. M65132 and M64928) NY-ESO-1 (GenBank Ace. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Ace. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad. Sci. USA, 91:3515 (1994); GenBank Ace. No. S73003, Adema, et al., J. Biol. Chem., 269:20126 (1994)), MAGE (van den Bruggen, et al., Science, 254:1643 (1991)); GenBank Ace. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Ace. No. U19180; U.S. Pat. Nos. 5,683,886 and 5,571,711), GAGE (GenBank Ace. Nos. AF055475, AF055474, AF055473, U19147, U19146, U19145, U19144, U19143 and U 19142), any of the CTA class of receptors including in particular HOM-MEL-40 antigen encoded by the SSX2 gene (GenBank Ace. Nos. X86175, U90842, U90841 and X86174), carcinoembryonic antigen (CEA, Gold and Freedman, J. Exp. Med., 121:439 (1985); GenBank Ace. Nos. M59710, M59255 and M29540), and PyLT (GenBank Ace. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including but not limited to MAGEA (CT1); BAGE (CT2); MAGEB (CT3); GAGE (CT4); SSX (CT5); NY-ESO-1 (CT6); MAGEC(CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pinel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed fusion proteins.

b. Antigens Associated with Tumor Neovasculature

Protein therapeutics can be ineffective in treating tumors because they are inefficient at tumor penetration. Tumor-associated neovasculature provides a readily accessible route through which protein therapeutics can access the tumor. In another embodiment the fusion proteins contain a domain that

specifically binds to an antigen that is expressed by neovasculature associated with a tumor.

The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed fusion proteins.

### III. Methods for Screening for Modulators of B7-H1 Function

Methods for identifying modulators of the function, expression, or bioavailability of B7-H1, homologues thereof, and receptors of B7-H1 utilize well known techniques and reagents. Representative receptors of B7-H1 include, but are not limited to PD-1 and non-PD-1 receptors. The modulator can modulate the B7-H1 signaling pathway, for example to inhibit or reduce B7-H1 signaling by interacting with B7-H1 or by interfering with B7-H1 binding to a B7-H1 receptor. Modulation of B7-H1 can be direct or indirect. Direct modulation refers to a physical interaction between the modulator and B7-H1 mRNA, protein, or DNA. Indirect modulation of B7-H1 can be accomplished when the modulator physically associates with a cofactor, second protein or second biological molecule that interacts with B7-H1 mRNA, DNA or protein either directly or indirectly. Additionally, indirect modulation includes modulators that affect the expression or the translation of RNA encoding B7-H1.

In some embodiments, the assays can include random screening of large libraries of test compounds. The test compounds are typically, non-protein small molecules. The term “small molecule” refers to compounds less than 1,000 daltons, typically less than 500 daltons. Alternatively, the assays may be used to focus on particular classes of compounds suspected of modulating the function or expression of B7-H1 or homologues thereof in cells, tissues, organs, or systems.

Assays can include determinations of B7-H1 expression, protein expression, protein activity, signal transduction, or binding activity. Other assays can include determinations of B7-H1 nucleic acid transcription or translation, for example mRNA levels, mRNA stability, mRNA degradation, transcription rates, and translation rates.

In one embodiment, the identification of a B7-H1 modulator is based on the function of B7-H1 in the presence and absence of a test compound. The test compound or modulator can be any substance that alters or is believed to alter the function of 87-H1, in particular the function of B7-H1 in the B7-H1 signaling pathway. Typically, a modulator will be selected that reduces, eliminates, or inhibits B7-H1 mediated signaling or increases or enhances apoptosis of cancer cells expressing B7-H1.

One exemplary method includes contacting B7-H1 protein with at least a first test compound, and assaying for an interaction between B7-H1 and the first test compound with an assay. The assaying can include determining inhibition of B7-H1 pro-apoptotic signaling, cell survivability, or lifespan assays.

Specific assay endpoints or interactions that may be measured in the disclosed embodiments include assaying for B7-H1 cell signaling or lifespan modulation, down or up regulation or turnover. These assay endpoints may be assayed using standard methods such as FACS, FACE, ELISA, Northern blotting and/or Western blotting. Moreover, the assays can be conducted in cell free systems, in isolated cells, genetically engineered cells, immortalized cells, or in organisms such as transgenic animals.

Other screening methods include using labeled B7-H1 to identify a test compound. B7-H1 can be labeled using standard labeling procedures that are well known and used in the art. Such labels include, but are not limited to, radioactive, fluorescent, biological and enzymatic tags.

Another embodiment provides a method for identifying a modulator of B7-H1 expression by determining the effect a test compound has on the expression of B7-H1 in cells. For example isolated cells or whole organisms expressing B7-H1 or both can be contacted with a test compound. B7-H1 expression can be determined by detecting LIN-14 protein expression or B7-H1 or B7-H1 mRNA transcription or translation. Suitable cells for this assay include, but are not limited to, cancer cells, immortalized cell lines, primary cell culture, or cells engineered to express B7-H1, for example cells from mammals such as humans. Compounds that modulate the expression of I B7-H1, in particular that reduce or inhibit B7-H1 cell signaling, can be selected.

Another embodiment provides for in vitro assays for the identification of B7-H1 modulators or modulators of B7-H1 homologues. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule, for example a nucleic acid encoding B7-H1 or B7-H1 protein, in a specific fashion is strong evidence of a related biological effect. Such a molecule can bind to a B7-H1 nucleic acid and modulate expression of B7-H1. The binding of a molecule to a target may, in and of itself, be inhibitory, due to steric, allosteric or charge—charge interactions or may down-regulate or inactivate B7-H1. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

Other embodiments include methods of screening compounds for their ability to modulate B7-H1 function or homologues thereof in cells. Various cell lines can be utilized for such screening assays, including cells specifically engineered for this purpose. Suitable cells include cancer cells that express B7-H1. Cells can also be engineered to express B7-H1 or a modulator thereof. Furthermore, those of skill in the art will appreciate that stable or transient transfections, which are well known and used in the art, may be used in the disclosed embodiments.

For example, a transgenic cell comprising an expression vector can be generated by introducing the expression vector into the cell. The introduction of DNA into a cell or a host cell is well known technology in the field of molecular biology and is described, for example, in Sambrook et al., Molecular Cloning 3rd Ed. (2001). Methods of transfection of cells include calcium phosphate precipitation, liposome mediated transfection, DEAE dextran mediated transfection, electroporation, ballistic bombardment, and the like. Alternatively, cells may be simply transfected with the disclosed expression vector using conventional technology described in the references and examples provided herein. The host cell can be a prokaryotic or eukaryotic cell, or any transformable organism that is capable of replicating a vector and/or expressing a heterologous gene encoded by the vector. Numerous cell lines and cultures are available for use as a host cell, and they can be obtained through the American Type Culture Collection (ATCC), which is an organization that serves as an archive for living cultures and genetic materials (www.atcc.org).

A host cell can be selected depending on the nature of the transfection vector and the purpose of the transfection. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (Stratagene, La Jolla, Calif.). Alternatively, bacterial cells such as E. coli LE392 could be used as host cells for phage viruses. Eukaryotic cells that can be used as host cells include, but are not limited to, yeast, insects and mammals. Examples of mammalian eukaryotic host cells for replication and/or expression of a vector include, but are not limited to, HeLa, NIH3T3, Jurkat, 293, Cos, CHO, Saos, and PC12. Examples of yeast strains include YPH499, YPH500 and YPH501. Many host cells from various cell types and organisms are available and would be known to one of skill in the art. Similarly, a viral vector may be used in conjunction with either an eukaryotic or prokaryotic host cell, particularly one that is permissive for replication or expression of the vector. Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

In vivo assays involve the use of various animal models, including non-human transgenic animals that have been engineered to have specific defects, or carry markers that can be used to measure the ability of a test compound to reach and affect different cells within the organism. Due to their size, ease of handling, and information on their physiology and genetic make-up, mice are a preferred embodiment, especially for transgenic animals. However, other animals are suitable as well, including C. elegans, rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more test compounds are administered to an animal, and the ability of the test compound(s) to alter one or more characteristics, as compared to a similar animal not treated with the test compound(s), identifies a modulator. Other embodiments provide methods of screening for a test compound that modulates the function of B7-H1. In these embodiments, a representative method generally includes the steps of administering a test compound to the animal and determining the ability of the test compound to reduce one or more characteristics of aging, lifespan, or age-related disorder.

Treatment of these animals with test compounds will involve the administration of the compound, in an appropriate form, to the animal. Administration will be by any route that could be utilized for clinical or non-clinical purposes, including, but not limited to, oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

### IV. Methods of Treatment

The disclosed compositions can be administered to a subject in need thereof to treat, alleviate, or reduce one or more symptoms associated with cancer or other forms of cellular hyperproliferation. In preferred embodiments, the compositions are administered in an amount effective to interfere, inhibit, or block B7-H1 signal transduction in cancer cells expressing B7-H1. The types of cancer that can be treated with the provided compositions and methods include, but are not limited to, the following: bladder, brain, breast, cervical, colorectal, esophageal, kidney, liver, lung, nasopharangeal, pancreatic, prostate, skin, stomach, uterine, ovarian, and testicular. Administration is not limited to the treatment of an existing tumor but can also be used to prevent or lower the risk of developing such diseases in an individual, i.e., for prophylactic use. Potential candidates for treatment include individuals with a high risk of developing cancer, i.e., with a personal or familial history of certain types of cancer.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

Administration

Administration of the polypeptide compositions described herein may be accomplished by any acceptable method which allows the polypeptide compounds, for example a B7-H1 antagonist, to reach its target. Because oral delivery of polypeptides is often unsuccessful due to the enzymes in the digestive tract, the disclosed polypeptide compositions are preferably administered parenterally. For small molecule antagonists of B7-H1 on tumor cells, the preferred method of administration is oral administration. The particular mode selected will depend of course, upon factors such as the particular formulation, the severity of the state of the subject being treated, and the dosage required for therapeutic efficacy. The actual effective amounts of polypeptide or small molecule compounds can vary according to the specific compound or combination thereof being utilized, the particular composition formulated, the mode of administration, and the age, weight, condition of the individual, and severity of the symptoms or condition being treated.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. The polypeptide or small molecule B7-H1 antagonist composition can be injected intradermally for treatment or prevention of cancer. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The formulations may be delivered using a bioerodible implant by way of diffusion or by degradation of the polymeric matrix. In certain embodiments, the administration of the formulation may be designed so as to result in sequential exposures to the antagonist over a certain time period, for example, hours, days, weeks, months or years. This may be accomplished, for example, by repeated administrations of a formulation or by a sustained or controlled release delivery system in which the antagonist is delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which the antagonist is contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the antagonist. In addition, a pump-based hardware delivery system may be used to deliver one or more embodiments.

Examples of systems in which release occurs in bursts includes, e.g., systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be e.g., in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

### V. Formulations

The compositions are administered to an individual in need of treatment or prophylaxis of at least one symptom or manifestation (since disease can occur/progress in the absence of symptoms) of cancer or cellular hyperproliferation. In one embodiment, the compositions are administered in an effective amount to inhibit B7-H1-mediated signal transduction in cancer cells expressing B7-H1. Alternatively, a composition is administered in an amount effective to increase, promote, or enhance apoptosis in cancer cells expressing B7-H1. The amount of inhibition of B7-H1 signal transduction or promotion of apoptosis in cancer cells can be determined relative to a control, for example cancer cells that are not treated with a B7-H1 antagonist. Methods for measuring inhibition of signal transduction are known in the art. Methods for measuring apoptosis are disclosed in the Examples.

The compounds are preferably employed for therapeutic uses in combination with a suitable pharmaceutical carrier. Such compositions include an effective amount of the compound, and a pharmaceutically acceptable carrier or excipient. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids some of which are described herein.

The compounds may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles or microspheres formed of biodegradable or non-biodegradable polymers or proteins or liposomes for targeting to cells. Such systems are well known to those skilled in the art and may be optimized for use with the appropriate nucleic acid.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, with an added preservative.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The compound alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. For administration by inhalation, the compounds are conveniently delivered in the form of an aerosol spray presentation from pressurized packs or a nebulizer, with the use of a suitable propellant.

In some embodiments, the compound described above may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the compounds are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett. 14(19):4975-4977 (2004)) and in vivo (Soutschek, et al., Nature 432(7014):173-178 (2004)). Other groups that can be attached or conjugated to the compounds described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; enzymes such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also described methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

Dosages

Dosages for a particular individual can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a individual is sufficient to effect a beneficial therapeutic response in the individual over time, or, e.g., to reduce symptoms, or other appropriate activity, depending on the application. The dose is determined by the efficacy of the particular formulation, and the activity, stability or serum half-life of the antagonist employed and the condition of the individual, as well as the body weight or surface area of the individual to be treated. The size of the dose is also determined by the existence, nature, and extent of any adverse side-effects that accompany the administration of a particular vector, formulation, or the like in a particular individual.

Formulations are administered at a rate determined by the LD50 of the relevant formulation, and/or observation of any side-effects of the compositions at various concentrations, e.g., as applied to the mass and overall health of the individual. Administration can be accomplished via single or divided doses.

In vitro models can be used to determine the effective doses of the compositions as a potential cancer treatment, as described in the examples. In determining the effective amount of the compound to be administered in the treatment or prophylaxis of disease the physician evaluates circulating plasma levels, formulation toxicities, and progression of the disease. For the disclosed compositions, the dose administered to a 70 kilogram individual is typically in the range equivalent to dosages of currently-used therapeutic antibodies such as Avastin®, Erbitux® and Herceptin®.

The formulations described herein can supplement treatment conditions by any known conventional therapy, including, but not limited to, antibody administration, vaccine administration, administration of cytotoxic agents, chemotherapy agents, natural amino acid polypeptides, nucleic acids, nucleotide analogues, and biologic response modifiers. Two or more combined compounds may be used together or sequentially. For example, the compositions can also be administered in therapeutically effective amounts as a portion of an anti-cancer cocktail. Anti-cancer cocktails can include therapeutics to treat cancer or angiogenesis of tumors.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### Expression of B7-H1 Confers Resistance of Tumor Cells to Specific CTL-Mediated Lysis

### Mice and Tumor Lines

Female DBA/2, C57BL/6 (B6) mice were purchased from the National Cancer Institute (Frederick, Md.). Age-matched mice, 6 to 10 weeks old, were used for all experiments. 2C transgenic mice (a gift from Dr. Larry Pease, Mayo Clinic, Rochester, Minn.) and PD-1 deficient mice in B6 background (a gift from Dr. Tasuko Honjo, Kyoto University, Kyoto, Japan) were described previously¹⁶. The 2C×PD-1KO mice were obtained by backcrossing between 2C transgenic mice and PD-1KO mice. All mice were maintained in the Animal Facility at Johns Hopkins Hospital under approved protocol by the Institutional Animal Care and Use Committee. P815 mastocytoma cells were purchased from the American Type Culture Collection (Rockville, Md.). A subclone, which does not express B7-H1, even in the presence of IFN-gamma or activated T cells, was selected before transfection. Stable transfectants of P815 lines including mock/P815 and B7-H1/P815 were described previously (Hirano, F. et al., Cancer Res., 65:1089-1096 (2005)). Renca is an H-2^{d} murine renal cell carcinoma line (a gift from Dr. Drew M. Pardoll, Johns Hopkins University).

### CTL Growth and Functional Assay

For the generation of alloreactive CTLs or 2C CTLs, lymph node cells (5×10⁶/ml) from C57BL/6 mice were stimulated with irradiated spleen cells (2×10⁶/ml) from DBA/2 mice in 24-well plates for 5 days. Cells were incubated with ⁵¹Cr-labeled target cells at the indicated effector/target (E/T) ratios for 4 hours. The spontaneous releases of ⁵¹Cr are <10%. In cold target competition assay, mock/P815 and B7-H1/P815 cells were premixed and incubated with activated 2C CTLs in 24-well plates for 4 hours. Cells were stained with PE-conjugated anti-H-2D^{d} mAb and 10B5 mAb plus FITC-conjugated goat anti-hamster immunoglobulin antibodies or GFP, and all cells were counted by flow cytometry. Wells containing only target cells were included as controls. To prepare PD-1⁰2C CTL clone, the lymph node cells from 2C×PD-1KO mice were maintained in the complete RPMI 1640 medium supplemented with 10% fetal bovine serum, 25 mM HEPES, 100 units/ml penicillin G, 100 μg/ml streptomycin sulfate, 55 μmol/L 2-ME and 30 units/ml human IL-2 by stimulating with 50 Gy-irradiated spleen cells from DBA/2 mice every 10-14 days.

### Plasmids

To generate truncated chimeric murine ΔB7-H1, full length B7-DC and PD-1, PCR fragments of each genes were digested with XhoI and BglII, XhoI and EcoRI or BamH1 and Xho1 restriction sites, respectively (5′ primer for truncated B7-H1; 5′-ccgctcgaggccaccatgaggatatttgctg-3′ (SEQ ID NO:1), 3′ primer for truncated B7-H1; 5′-gaagatcttcttgttttctcaagaaga-3′ (SEQ ID NO:2), 5′ primer for full length B7-DC; 5′-ccgctcgaggccaccatgctgctcctgctgccga-3′ (SEQ ID NO:3), 3′ primer for full length B7-DC; 5′-ggaattcctagatcctctttctct-3′(SEQ ID NO:4), 5′ primer for full length PD-1; 5′-cgcggatccgccaccatgtgggtccggcag-3′(SEQ ID NO:5) and 3′ primer for full length PD-1; 5′-ccgctcgagtcaaagaggccaagaac-3′ (SEQ ID NO:6). Subsequent ligations of these fragment were performed into XhoI/BamHI-digested pEGFP-N1 (Clontech, Palo Alto, Calif.), XhoI/EcoRI-digested pcDNA3.1 (Invitrogen, Carlsbad, Calif.) or BamHI/XhoI-digested pHR' CMV vectors. Chimeric murine B7-DC/H1 and truncated chimeric murine ΔPD-1 was constructed by two-step PCR. Overlapping oligonucleotide primers were synthesized and 2 flanking 5′ and 3′ primers were designed to contain XhoI and BglII or BamHI and XhoI restriction sites. Appropriate regions of cDNA were initially amplified using the corresponding overlapping and flanking primers (5′ primer for B7-DC; 5′-ccgctcgaggccaccatgctgctcctgctgccga-3′ (SEQ ID NO:7), 3′ primer for B7-DC; 5′-cagaagcacccagtgccacgttctggggac-3′ (SEQ ID NO:8), 5′ primer for B7-H1; 5′-gtccccagaacgtggcactgggtgcttct-3′(SEQ ID NO:9), 3′ primer for B7-H1; 5′-gaagatattacgtctcctcgaattgtgt-3′ (SEQ ID NO:10), 5′ primer for PD-1; 5′-cgcggatccgccaccatgtgggtccggcag-3′ (SEQ ID NO:11), 3′ primer for PD-1; 5′-gcccttgctcaccatcttgttgagcagaagac-3′(SEQ ID NO:12), 5′ primer for EGFP; 5′-cttctgctcaacaagatggtgagcaagggc-3′ (SEQ ID NO:13) and 3′ primer for EGFP; 5′-ccgctcgagttacttgtacagctcgtc-3′ (SEQ ID NO:14)). Then, using the flanking 5′ and 3′ primers, fragments whose sequences overlapped were fused together and amplified. PCR products were digested with XhoI and BglII or BamHI and XhoI and ligated into XhoI/BamHI-digested pcDNA3.1 or BamHI/XhoI-digested pHR' CMV vectors.

### Gene Transfection and Lentivirus-Mediated Transduction

To generate ΔB7-H1/P815, B7-DC/P815 and B7-DC/H1/P815, P815 cells were transfected with corresponding vectors by using electroporation and cloned by limiting dilution as described previously. Recombinant lentiviruese with full length or truncated PD-1 were used to transduced 2C×PD-1KO CTLs, The 293T cell lines were cultured in Dulbecco's modified

Eagle's medium supplemented with 10% fetal calf serum. All recombinant lentiviruses were produced by transient transfection of 293T cells according to standard protocols. Briefly, subconfluent 293T cells were cotransfected with 20 μg of a plasmid vector, 15 μg of pCMV-ΔR8.91, and 5 μg of pMD2G-VSVG by lipofectamine (Invitrogen, San Diego, Calif.). After 48 h recombinant lentivirus vectors were harvested. For transduction, 2C×PD-1KO T cells were plated on 24-well plate, and RPMI medium containing 50% medium containing recombinant lentivirus vectors, 10% fetal bovine serum, 25 mM HEPES, 100 units/ml penicillin G, 100 μg/ml streptomycin sulfate, 55 μmol/L 2-ME and 30 units/ml human IL-2 was added. Following 48 hr of incubation, the cells were washed and split. The procedure was repeated for 4 times to increase transduction efficiency. PD-1 positive cells after transduction were purified by magnetic beads (Miltenyi Biotec, Auburn, Calif.).

### Results

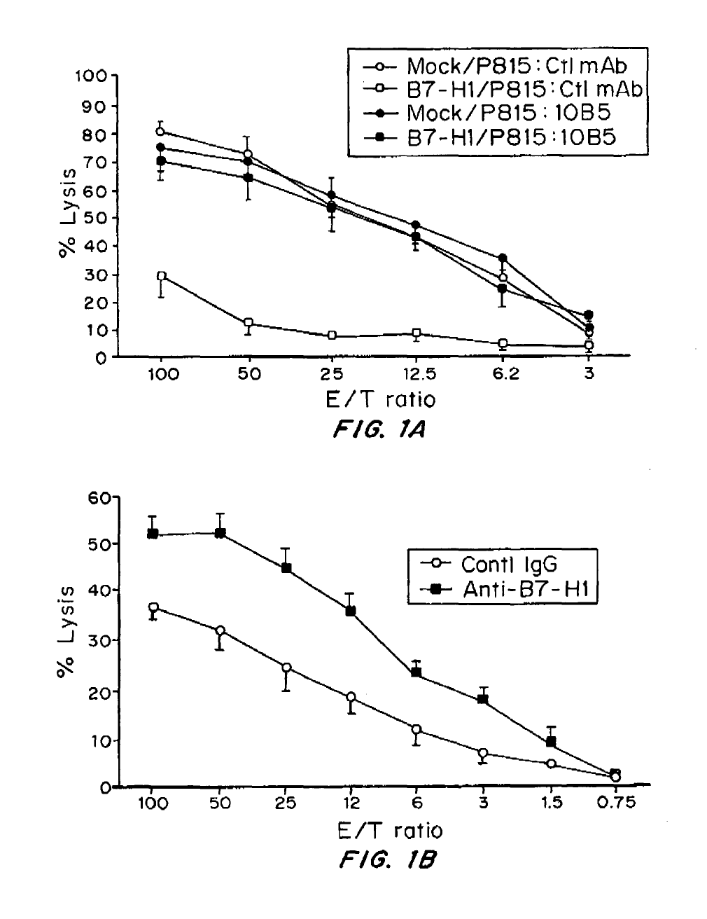

Previous studies showed that expression of B7-H1 rendered P815 cells resistant to tumor-specific CD8+ CTL and this resistance requires B7-H1 and PD-1 interaction (Hirano, F., et al., Cancer Res., 65:1089-1096 (2005)). To determine whether this observation could be generalized, an allogeneic CTL line was established by stimulation of B6-derived T cells with irradiated spleen cells from DBA/2 mice (H-2^{d}). The susceptibility of mock/P815 and B7-H1/P815 cells to lysis in a 4-hr ⁵¹Cr release assay was determined. As predicted, while mock/P815 cells were susceptible to lysis by allogeneic CTL in a wide range of effector/target ratios, B7-H1/P815 cells were much more resistant. This resistance, however, could be completely eliminated by inclusion of murine B7-H1 neutralizing mAb. (FIG. 1A). Next Renca cells were used as targets for lysis by alloantigen-specific TCR transgenic 2C T cells. Renca is a murine renal cell carcinoma and does not constitutively express B7-H1 on the cell surface. However, expression of B7-H1 could be induced by IFN-gamma (data not shown). After incubation with mouse interferon-gamma (5 ng/ml) for 48 hr, Renca cells were co-cultured with pre-activated 2C T cells for 4 hrs in the presence of control or anti-murine B7-H1 mAb (clone10B5). 10B5 significantly increased lysis of 2C CTLs against Renca cells in a wide range of effector:target ratios (from 3:1 to 100:1) (FIG. 1B), Taken together, the data indicate that B7-H1-mediated “molecular shield” is not limited to a specific T cell or certain tumor lines.

### Example 2

### PD-1 Signaling is not Required for Molecular Shielding of Tumor Cells from T Cell Killing

A previous study showed that interaction between B7-H1 and PD-1 is required for the formation of a molecular shield (Hirano, F., et al. Cancer Res., 65:1089-1096 (2005)). It is widely accepted that signaling from B7-H1 to PD-1 on T cells delivers a negative signal to suppress T cell responses (Freeman, G. J., et al. J Exp Med., 192:1027-1034 (2000)). Therefore, one possible explanation for molecular shield is tumor-associated B7-H1 signaling through PD-1 into T cells, leading to transient loss of T cell cytolytic activity. To test this possibility, a truncated PD-1 was prepared as described in Example 1, in which the intracellular domain of PD-1 was replaced by green fluorescence protein (GFP) gene (FIG. 2A) to eliminate its intracellular signaling. This truncated PD-1 (ΔPD-1) as well as wild type PD-1 (PD-1) was inserted into lentiviral vectors for efficient T cell transduction. To eliminate interference of endogenous PD-1 on T cells, 2C T cells were backcrossed to PD-1KO mice (2C×PD-1KO) and established PD-1KO (PD-1⁰2C CTLs clone from 2C×PD-1KO mice. A PD-1⁰2C CTL clone was transduced with the recombinant PD-1 lentiviruses containing either ΔPD-1 or PD-1 to establish CTL lines. Both PD-1/PD-1⁰2C and ΔPD-1/PD-1⁰2C lines which stably expressed cell surface PD-1 were established. Both 2C lines express high level cell surface PD-1 (FIG. 2B). Although ΔPD-1/PD-1⁰2C expresses somewhat higher levels of cell surface PD-1 than PD-1/PD-1⁰2C, their cytolytic activity against mock/P815 cells are comparable to 2C CTL from PD-1KO mice (FIG. 2C). This result indicates that cytolytic activities of these CTL lines are indistinguishable in the absence of B7-H1 on tumor cells. When B7-H1/P815 cells were used as targets, cytolytic activity of PD-1/PD-1⁰2C CTLs was decreased to approximately 50% in comparison with PD-1⁰2C T cells. Interestingly, ΔPD-1/PD-1⁰2C CTLs demonstrated a virtually identical decrease in cytolytic activity as PD-1⁰2C T cells (FIG. 2D). Therefore, the intracellular domain of PD-1 is not required for the resistance to CTL lysis. The results thus support that PD-1 signaling to T cells does not contribute to the molecular shield and suggest a possible role for reverse signaling by B7-H1 into the tumor cell as a mechanism of molecular shielding.

### Example 3

### Intracellular Domain of B7-H1 is Required for Molecular Shielding

### Monoclonal Antibodies, Fusion Proteins and Flow Cytometry Analysis

Purified mAb against mouse H-2D^{d} and CD95 (clone Jo2) was purchased from PharMingen (San Diego, Calif.) and H-2L^{d} from BioLegend (San Diego, Calif.). Rat mAb (clone TY25) against B7-DC, FITC- or phycoerythrin-conjugated goat anti-mouse antibodies and FITC-conjugated goat anti-hamster antibodies were purchased from eBioseience (San Diego, Calif.). An Armenian hamster mAb (clone 10135) against mouse B7-H1 (Hirano, F. et al., Cancer Res., 65:1089-1096 (2005)), a hamster mAb (clone G4) against mouse PD-1 (Hirano, F. et al., Cancer Res., 65:1089-1096 (2005)), a rat mAb (clone 2A) against mouse CD137 (Wilcox, R. A., et al., J Clin Invest., 109:651-659 (2002)), mouse PD-1Ig fusion protein (Hirano, F. et al., Cancer Res., 65:1089-1096 (2005)) and mouse B7-H1Ig fusion protein (Hirano, F. et al., Cancer Res., 65:1089-1096 (2005)) were all described previously. Fluorescence was detected by FACScalibur flow cytometry and analyzed with Cell Quest software (Becton Dickinson, Mountain View, Calif.).

### Results

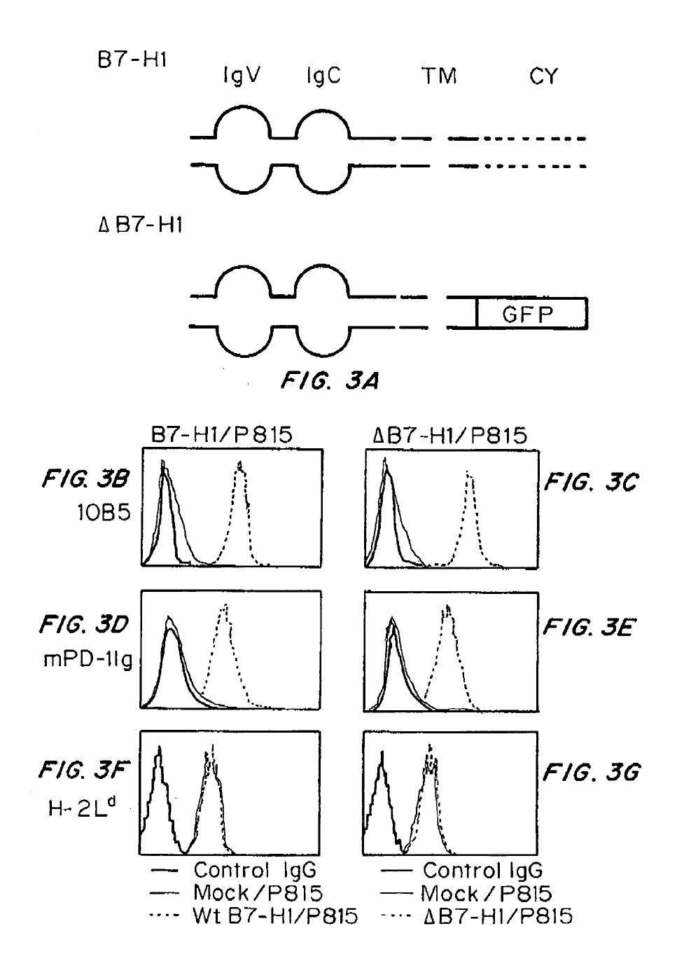

To determine the role of tumor-associated B7-H1 signaling in the formation of the molecular shield, a vector was constructed as described in Example 1 to express truncated B7-H1 (ΔB7-H1), in which the intracellular domain of B7-H1 was replaced by GFP gene (FIG. 3A). A stable P815 line expressing ΔB7-H1 was established and the expression of B7-H1 and H-2 L^{d} (FIGS. 3B-G) as well as the ability to bind PD-1Ig fusion protein or anti-B7-H1 antibody are comparable to the line expressing wild type B7-H1 (B7-H1) (FIGS. 3B-G). Both ΔB7-H1/P815 and B7-H1/P815 cells could inhibit the proliferation of T cells in vitro (data not shown). These data indicate that ΔB7-H1 has normal binding to PD-1 and induces functional PD-1 in T cells. P815 cells were assayed to determine whether expressing ΔB7-H1 could still confer tumor resistance to CTL lysis. While B7-H1/P815 cells were resistant to lysis by 2C T cells, ΔB7-H1/P815 cells lost resistance and were lysed equally as mock/P815 cells by 2C T cells. In addition, the inclusion of 10B5 did not affect the lysis (FIG. 3H), indicating that the intracellular domain of B7-H1 is critical for the formation of molecular shield. To validate this, 1:1 mixes of mock/P815:B7-H1/P815, mock/P815:ΔB7-H1/P815 and B7-H1/P815:ΔB7-H1/P815 were prepared followed by coincubated with 2C CTLs for 4 hr, as described previously. The cytolysis for each cell was analyzed by flow cytometry. The ratio of B7-H1/P815 vs. mock/P815, B7-H1/P815 vs. ΔB7-H1/P815 and ΔB7-H1/P815 vs. mock/P815 were 6.51, 9.50 and 1.15, respectively. These results indicate that ΔB7-H1, similar to mock/P815, fails to confer molecular shielding to P815 and could not induce cytolytic function of 2C T cells in vitro.

While previous data suggest that intracellular domain of B7-H1 is important in determining the formation of molecular shield, it could not be excluded that the extracellular domain of B7-H1 may also contribute to the formation of molecular shield. To address this issue, the extracellular domain of B7-H1 was replaced with corresponding region from B7-DC, another counter-receptor for PD-1 (Latchman, Y. et al., Nat. Immunol., 2:261-268 (2001)). (FIG. 4A). The chimeric gene, B7-DC/H1, was used for the transfection to establish stable B7-DC/H1/P815 line. P815 cells expressing wild type B7-DC (B7-DC/P815) were also established as the control. The expression of B7-DC and H-2 L^{d} (data not shown) as well as ability of these cell lines to bind PD-1Ig was the same in both B7-DC/P815 and B7-DC/H1/P815 lines based on flow cytometry analysis (FIG. 4B). As shown in FIG. 4F, B7-DC/P815 cells could be lysed equally as well as mock/P815 cells by 2C T cells. This result indicates that B7-DC is not capable of forming a molecular shield even though B7-DC is capable of engaging PD-1 on T cells with affinity at least as high as B7-H1. In contrast, B7-DC/H1/P815 cells were resistant to 2C CTLs and this resistance was completely blocked by anti-B7-DC mAb (clone TY25). The findings thus demonstrate that intracellular but not extracellular domain of B7-H1 has the unique ability to form a molecular shield against T cell lysis.

### Example 4

### Formation of Molecular Shield is Correlated with Tumor Resistance to Immunotherapy

Reverse signaling by B7-H1 was evaluated to determine whether it conferred resistance to immune killing of tumors in vivo. It was shown previously that B7-H1/P815 tumors were resistant to the treatment by anti-CD137 mAb while mock/P815 or wild type P815 were sensitive in vivo (Hirano, F., et al., Cancer Res., 65:1089-1096 (2005)). Anti-CD137 immunotherapy of tumors has been shown to be due to enhanced stimulation of endogenous tumor-specific CD8 T-cells in vivo. Because ΔB7-H1/P815 fails to be shielded from CTL lysis in vitro, resistance of this line to CD137 mAb therapy in vivo was assessed. To do so, ΔB7-H1/P815 cells were inoculated into syngeneic DBA/2 mice to induce progressive growth of subcutaneous tumors. Mock/P815 and B7-H1/P815 cells were also inoculated into a group of mice as controls. As expected, all tumors treated with control mAb grew equally well and eventually killed the mice because growth of P815 tumor itself is not sufficient to induce significant anti-tumor immunity. However, treatment by anti-CD137 mAb (clone 2A) induced complete regression of mock/P815 tumors after a transient growth period while B7-H1/P815 tumors continued to grow and eventually killed the mice. These data indicate that B7-H1-mediated molecular shielding operates to prevent immune attack in vivo. In contrast, P815 cells expressing ΔB7-H1 regressed completely, a result identical to Mock/P815 (FIG. 5). The results thus further support that the intracellular domain of B7-H1 is required for the formation of a molecular shield, which correlates with tumor resistance in vivo.

### Example 5

### B7-H1 Transmits an Anti-Apoptotic Signal to Tumor Cells

### Cell Apoptosis Assays

PD-1Ig fusion protein or control IgG were coated on 96 well-plates for 18 hr at 4° C. Full length B7-H1/P815 cells were cultured with STP (1 μM) in the well coated PD-1Ig or control IgG. Cells were harvested after 24 hr and stained with annexin V (BD PharMingen) at 5 μl/test, propidium iodide (PI) (Sigma-Aldrich) at 5 μg/ml for 1 hour and monoclonal antibodies against H-2D^{d}. Apoptosis was calculated as the percentage of annexin V+PI-cells gated in the H-2D^{d}+ fractions.

### Results

To explore the mechanism of molecular shielding by B7-H1 signaling, a T cell-free system was developed to induce resistance of tumor cells to CTL lysis, In this system, PD-1Ig was immobilized on plastic plates and subsequently incubated with tumor cells to engage B7-H1. Twenty-four hrs later, tumor cells were incubated with PD-1⁰2C T cells to test susceptibility of tumor cells to lysis. After 4 hrs incubation, the lysis of PD-1Ig-treated B7-H1/P815 cells by PD-1⁰2C T cells was significantly lower than control IgG-treated B7-H1/P815 cells in a wide range of E/T ratios (FIG. 6A). This provides a simple system to explore the mechanism of B7-H1-induced molecular shielding in the absence of T cells in the induction phase.

Fas has been shown to participate in the death of some cancer cells. Mock/P815 and B7-H1/P815 cells express comparable levels of Fas on the cell surface based on flow cytometry analysis using specific mAb against murine Fas. Incubation of these tumor lines in the presence of PD-1Ig does not affect expression of Fas (FIGS. 6B and C). Treatment of mock/P815 and B7-H1/P815 cells by anti-Fas mAb (and control Ig) also induced a comparable amount of apoptosis (54.9% vs. 57.2%). However, pretreatment by PD-1Ig significantly decreased the death of B7-H1/P815 cells (35.8%) while the death of mock/P815 cells remained the same (52.4%) (FIGS. 6D-H). This represents 32% inhibition.

B7-H1 was assayed to determine whether it confers resistance to drugs which induce apoptosis of tumor cells. Staurosporine (STP) is (Streptomyces staurospores) is a relatively non-selective protein kinase inhibitor. Staurosporine is often used as a general method for inducing apoptosis of tumor cells. PD-1Ig pretreatment for 8 hrs drastically decreased apoptosis of B7-H1/P815 cells (16.9%) in comparison with control Ig-treated cells (74.9%). Taken together, the results support that B7-H1 is an anti-apoptotic receptor that inhibits the death of cancer cells.

1. (canceled)

2. (canceled)

3. (canceled)

4. (canceled)

5. A method for increasing cancel cell death in a subject comprising locally administering to the cancer cells an effective amount of a modulator to interfere, inhibit or reduce B7-H1-mediated signal transduction in the cancer cells.

6. The method of claim 5, wherein the modulator is an antibody or antibody fragment that binds to an epitope on the extracellular domain of B7-H1 and inhibits or reduces B7-H1-mediated signal transduction in the cancer cells.

7. The method of claim 5, wherein the cancel cell death is due to apoptosis.

8. The method of claim 5, further comprising administering one or more additional therapeutic agents.

9. The method of claim 8, wherein the one or more additional therapeutic agents are selected from the group consisting of chemotherapeutic agents and pro-apoptotic agents.

10. The method of claim 5, wherein the cancer cells are selected from the group consisting of bladder, brain, breast, cervical, colorectal, espophageal, kidney, liver, lung, nasopharangeal, pancreatic, prostate, skin, stomach, uterine, ovarian, and testicular.

11. (canceled)

12. (canceled)

13. (canceled)

14. (canceled)

15. (canceled)

16. The method of claim 5 further comprising administering a modulator that binds PD-1, B7-1 or B7-H1 in an amount effective to inhibit or reduced binding of PD-1, B7-1 or both to B7-H1 expressed on tumor cells.

17. The method of claim 5, wherein the modulator comprises a soluble PD-1, B7-1, or B7-H1 polypeptide.

18. The method of claim 16, wherein the modulator comprises an antibody or antigen-binding fragment thereof.

19. The method of claim 5 further comprising administering antigen specific autologous T cells expanded in vitro where the antigen specific autologous T cells are specific for a tumor associated or tumor specific antigen expressed by the cancer cells.

20. A method for increasing cancer cell death in a subject comprising administering to the cancer cells an effective amount of a soluble PD-1 polypeptide to promote apoptosis or increase susceptibility to apoptosis of the cancer cells.
